(19) United States
(12) Patent Application Publication
(10) Pub. No.: US 2026/0027179 A1
(43) Pub. Date: Jan. 29, 2026

(54) ANTI-SYNUCLEINOPATHY PEPTIDE AND METHODS TO TREAT NEURODEGENERATIVE DISEASES

(71) Applicant: University of British Columbia, Vancouver (CA)

(72) Inventors: Yu Tian Wang, Vancouver (CA); Jack Jin, Vancouver (CA); Xuelai Fan, Vancouver (CA)

(73) Assignee: University of British Columbia, Vancouver, BC (CA)

(21) Appl. No.: 18/276,087

(22) PCT Filed: Feb. 8, 2022
(86) PCT No.: PCT/CA2022/050175

§ 371 Date: Aug. 7, 2023

Related U.S. Application Data
(60) Provisional application No. 63/147,078, filed on Feb. 8, 2021.

(51) Int. Cl. A61K 38/17

(52) U.S. Cl. CPC A61K 38/1709

(57) ABSTRACT

Disclosed is a method of treating a neurodegenerative disease such as Parkinson's disease, diffuse Lewy body disease, transitional Lewy body dementia, and multiple system atrophy in a subject. The method comprises administering to the subject a therapeutically effective amount of a peptide comprising an α-synuclein binding domain operably linked to a protein transduction domain and a proteasomal targeting domain, wherein the α-synuclein binding domain is derived from a reversed sequence of β-synuclein. Other methods, as well as uses and compositions, are disclosed.

### CROSS-REFERENCE TO RELATED APPLICATION

This application claims priority to U.S. Provisional Patent Application Ser. No. 63/147,078 filed on Feb. 8, 2021 entitled “ANTI-SYNUCLEINOPATHY PEPTIDE AND METHOD OF USING SAME,” the contents of which are incorporated herein by reference.

### FIELD OF THE INVENTION

In one of its aspects, the present invention relates to methods of altering protein expression and aggregation, and in particular to proteasome-dependent, peptide-mediated knockdown of α-synuclein.

### BACKGROUND TO THE DISCLOSURE

Synucleinopathies such as Parkinson's disease (PD), dementia with Lewy bodies (DLB), and multiple system atrophy (MSA) represent a major group of neurodegenerative disorders that currently lack clinically relevant treatments capable of directly targeting the disease-causing processes. Current clinical approaches, like deep brain stimulation and pharmacological treatments with levodopa and dopamine agonists, only relieve symptoms. The efficacy of these treatments is largely limited by their undesirable complications and side effects.

Accumulating evidence supports the theory that the main symptoms of synucleinopathies are at least partly associated with α-synuclein protein aggregation in the cytoplasm of relevant cell populations, such as nigral dopaminergic neurons in PD. In addition, a growing body of evidence suggests that misfolded α-synuclein can propagate from one infected cell to an adjacent healthy cell in a prion-like manner and induce α-synuclein aggregation and cell dysfunction in the newly infected cell (see, e.g., Luk et al., J. Exp. Med. (2012), 209, 5, 975-986, www.jem.org/cgi/doi/10.1084/jem.20112457).

Knockdown of α-synuclein using genetic manipulations, such as anti-sense oligonucleotide and siRNA, has shown protection of dopaminergic neurons in various models of PD. The clinical translation of these genetic manipulations into an efficient PD therapy has, however, been hindered, at least in part, due to their limited ability to cross the blood brain barrier (BBB) and the plasma membrane of neurons in the affected areas of the brain. The delivery of siRNAs to the brain is mainly accomplished by an invasive intracerebral injection or viral infection, which may not be clinically practical for the therapeutic use in human patients. Several recent studies suggest that delivery of siRNA to the brain by a non-invasive systemic injection may be achieved by coupling siRNA with brain delivery vehicles such as RVG-9R peptide or RVG-9R peptide-coated exosome. Although recent studies suggest that this may be partially improved by coupling siRNA with a brain delivery vehicle, these techniques either are restricted to the acetylcholine receptor-expressing neurons in the brain or remain technically challenging and thus may not represent a practical solution for therapeutic use in human patients.

The development of a successful therapeutic with effect on disease-causing processes has also been hindered until recently by a lack of pathologically relevant animal models. Toxicity-based in vivo models such as the MPTP mouse model (and its in vitro counterpart, the MPP+ toxicity model in cultured midbrain neurons) do not produce the α-synuclein aggregation that is a pathological hallmark of PD and other synucleinopathies. As a result, data collected in such models may not accurately predict how a candidate therapeutic will perform in a more pathologically relevant animal model, much less in clinical use. For example, Levodopa/Benserazide, a clinically approved drug for PD, does not show desired protective effects in the MPTP model (Gevaerd et al, International Journal of Neuropsychopharmacology (2001), 4,361-370, DOI: 10.1017/S1461145701002619).

The recently developed pre-formed fibril model of spreading synucleinopathy (Luk et al. 2012, supra) offers a more pathologically and phenotypically relevant platform for the evaluation of candidate anti-synucleinopathy therapeutics. In this model, synthetic α-synuclein aggregates can “seed” and propagate a PD-like pathology in young asymptomatic M83 transgenic (Tg) mice, which express human α-synuclein with the known familial PD-related A53T mutation. As compared to the older toxicity-based models, this approach better reflects current mechanistic understanding, including the cell-to-cell propagation that is increasingly believed to underlie the progression of synucleinopathies such as PD, DLB, and MSA (Karpowicz et al., Lab Invest. (2019), 99, 7, 971-981, doi:10.1038/s41374-019-0195-z). As such, efficacy in this model may be more reliably predictive of therapeutic utility than previous models.

Despite the advances made to date in the development of therapeutics for Parkinson's disease and other synucleinopathies, there is room for improvement.

### SUMMARY OF THE INVENTION

It is an object of the present disclosure to obviate or mitigate at least one of the above-mentioned disadvantages of the prior art.

It is another object of the present disclosure to provide a novel peptide-based treatment for synucleinopathies such as Parkinson's disease.

Accordingly, in one of its aspects, the present disclosure provides a method of treating a disease characterized by abnormal aggregation of α-synuclein in a subject, the method comprising administering to the subject a therapeutically effective amount of a peptide comprising an α-synuclein binding domain operably linked to a protein transduction domain and a proteasomal targeting domain, wherein the α-synuclein binding domain is derived from a reversed sequence of β-synuclein.

In another aspect, the present disclosure provides a method of reducing neuroinflammation in a subject having a disease that is, or is characterized by, a synucleinopathy, the method comprising administering to the subject a therapeutically effective amount of a peptide comprising an α-synuclein binding domain operably linked to a protein transduction domain and a proteasomal targeting domain, wherein the α-synuclein binding domain is derived from a reversed sequence of β-synuclein.

In another aspect, the present disclosure provides a method of reducing the cell-to-cell propagation of α-synuclein in the brain of a subject having a disease that is, or is characterized by, a synucleinopathy, the method comprising administering to the subject a therapeutically effective amount of a peptide comprising an α-synuclein binding domain operably linked to a protein transduction domain and a proteasomal targeting domain, wherein the α-synuclein binding domain is derived from a reversed sequence of β-synuclein.

In another aspect, the present disclosure provides a use of a therapeutically effective amount of a peptide comprising an α-synuclein binding domain operably linked to a protein transduction domain and a proteasomal targeting domain, wherein the α-synuclein binding domain is derived from a reversed sequence of β-synuclein, for the treatment of a subject having a disease characterized by the abnormal aggregation of α-synuclein.

In another aspect, the present disclosure provides a use of a therapeutically effective amount of a peptide comprising an α-synuclein binding domain operably linked to a protein transduction domain and a proteasomal targeting domain, wherein the α-synuclein binding domain is derived from a reversed sequence of β-synuclein, to reduce the cell-to-cell propagation of α-synuclein in a subject having a disease that is, or is characterized by, a synucleinopathy.

In another aspect, the present disclosure provides a use of a therapeutically effective amount of a peptide comprising an α-synuclein binding domain operably linked to a protein transduction domain and a proteasomal targeting domain, wherein the α-synuclein binding domain is derived from a reversed sequence of β-synuclein, to reduce neuroinflammation in a subject having a disease that is, or is characterized by, a synucleinopathy.

In another aspect, the present disclosure provides a pharmaceutical composition for administration to a subject having a disease that is, or is characterized by, a synucleinopathy, the pharmaceutical composition comprising (a) a therapeutically effective amount of a peptide comprising an α-synuclein binding domain operably linked to a protein transduction domain and a proteasomal targeting domain, wherein the α-synuclein binding domain is derived from a reversed sequence of β-synuclein; and (b) a carrier.

Other advantages will become apparent to those of skill in the art upon reviewing the present specification.

### BRIEF DESCRIPTION OF THE DRAWINGS

Embodiments will be described with reference to the accompanying drawings, wherein like reference numerals denote like parts, and in which:

FIG. 1 illustrates the design of α-synuclein knockdown mini-genes and peptides and demonstration of knockdown in HEK 293 cells. (a) Schematic illustration of the Tat-βsyn-degron peptide design. The α-synuclein targeting peptide Tat-βsyn-degron has three domains: 1) the Tat transduction domain that enables the peptide to penetrate cell membranes, 2) the α-synuclein binding domain derived from β-synuclein, and 3) the degron sequence that targets the Tat-βsyn-degron and α-synuclein complex to the proteasome for degradation. In contrast, the Tat-βsyn control peptide lacks the proteasomal targeting signal, and hence while it can bind to α-synuclein, it cannot direct the complex to the proteasome for degradation. (b) Schematic illustration of the mini-gene constructs encoding FLAG-βsynN-degron (βsynN: natural amino acid sequence between 36-45 of β-synuclein), FLAG-βsyn-degron (βsyn: reversed β-synuclein 36-45) or FLAG-βsyn. (c-e) Immunoblots sequentially probing for α-synuclein and β-actin (as loading and specificity controls) showing that expression of FLAG-βsynN-degron (c; N=9; F(3,32)=3.43; P<0.05) or FLAG-βsyn-degron (d; N=5; F(3,16)=13.18; P<0.001), but not control FLAG-βsyn (e; N=4; F(3,12)=0.73; P=0.55), induced a dose-dependent reduction in the levels of human α-synuclein co-expressed in HEK 293 cells. Note that FLAG-βsyn-degron appears to have a better efficacy in reducing α-synuclein. Transfection ratios of the plasmids are shown on the top. (f) FLAG-βsyn-degron mediated knockdown is α-synuclein specific. Immunoblots sequentially probing for synuclein and β-actin showing that when co-transfected at 4:1 ratio in HEK 293 cells, FLAG-βsyn-degron, not FLAG-βsyn, specifically reduces the level of α-synuclein (N=7; F(2,18)=28.51; P<0.001), but not the levels of HA-β-synuclein (N=7; F(2,18)=0.42; P=0.66) or HA-γ-synuclein (N=7; F(2,18)=0.93, P=0.41). Data are presented as mean±S.E.M. The statistical difference between groups was determined by one-way ANOVA, followed by Bonferroni post hoc test. *P≤0.05 and **P≤0.01 and ***P≤0.001 compared with the control. n.s. denotes not significant.

FIG. 2 illustrates Biacore peptide-protein binding assays. (a-d). Representative sensorgrams demonstrating the binding responses of the synthetic Tat peptide (b), Tat-βsynN-degron peptide (c), Tat-βsyn-degron peptide (d), or HBS blank buffer control (a) to α-synuclein. Two-fold serial dilutions (0.20 μM, 0.39 μM, 0.78 μM, 1.56 μM, 3.13 μM, 6.25 μM, 12.50 μM) of peptides, in duplicate, were sequentially injected over immobilized purified recombinant human α-synuclein for 3 minutes, followed by a dissociation phase during which HBS buffer was flowed over the surface. Sensorgrams, depicting binding responses over time, were double-referenced by subtracting out the binding on the reference surface and the response from the HBS blank buffer control. Peptide—α-synuclein binding response report points were collected 20 seconds into the dissociation phase at time 200 seconds (as indicated by the vertical lines in the figures), to exclude bulk refractive index changes and nonspecific binding. (e) Graphing of peptide—α-synuclein binding response versus peptide concentration showing that synthetic Tat-βsynN-degron and Tat-βsyn-degron peptides displayed robust and similar binding with α-synuclein in a dose-dependent manner (0.20 μM, 0.39 μM, 0.78 μM, 1.56 μM, 3.13 μM, 6.25 μM, 12.50 μM), while the control Tat peptide displayed little binding with α-synuclein.

FIG. 3 illustrates a dose- and time-dependent knockdown effect of Tat-βsyn-degron peptide α-synuclein without significantly affecting the levels of several other cellular proteins. (a) Immunoblots showing that bath application of the membrane permeant synthetic peptide Tat-βsyn-degron at various concentrations for 24 hrs induced a robust reduction of endogenous α-synuclein protein levels in a dose-dependent manner (5-50 μM; N=9; F(6,56)=5.15; P<0.001); this was prevented in the presence of the proteasome inhibitor MG132 (Tukey's HSD post hoc test: 50 μM Tat-βsyn-degron+10 μM MG132; ##P<0.01, compared with the 50 μM Tat-βsyn-degron group; N=9). In contrast, bath application of the control Tat-βsyn had no effect (50 μM, 24 hrs; Tukey's HSD post hoc test: P=0.87 compared with the control; N=9). (b) Bath application of Tat-βsyn-degron (50 μM) induced a time-dependent knockdown of endogenous α-synuclein in primary cortical cultures (N=8; F(5,42)=8.06; P<0.001). (c-e) Tat-βsyn-degron at a high dose (50 μM; 24 hrs) did not affect the expression levels of several un-related proteins in cortical cultures. These proteins include transmembrane protein β2/3 subunit of the GABA_{A} receptor (c; N=3; F(5,12)=0.90; P=0.51), cytosol chaperone protein HSP90 (d; N=6; F(5,30)=1.33; P=0.28), and 14-3-3, a known α-synuclein binding protein (e; N=6; F(5,30)=0.43; P=0.82). (f) ELISA analysis showing decreased α-synuclein levels at 12 hrs and 24 hrs in the brain of M83 transgenic mice after an intraperitoneal injection of Tat-βsyn-degron (N=5 per group, F(4,20)=5.24, P<0.01). Data are presented as mean±S.E.M. The statistical difference between groups was determined by one-way ANOVA, followed by Tukey's HSD post hoc test. *P≤0.05 and **P<0.01 and ***P≤0.001 compared with the control. n.s. denotes not significant.

FIG. 4 illustrates a protective effect of Tat-βsyn-degron peptide against parkinsonian toxin induced neuronal damage in rat ventral midbrain cultures. Immunoblotting (a-c) and immunocytochemical staining (d) followed by blinded TH-positive neuron counting (e) showing that bath application (25 μM; 48 hrs) of Tat-βsyn-degron, but not Tat-βsyn, significantly reduced the level of endogenous α-synuclein (a and b; N=8; F(3,21)=18.39, P<0.001; Bonferroni post hoc test: MPP++Tat-βsyn-degron vs MPP+: ###P<0.001), and prevented MPP+(20 μM; 48 hrs)-induced TH-positive neuronal damage as demonstrated by the loss of TH protein (a and c; N=8; F(3,21)=6.87, P<0.01; Bonferroni post hoc test: MPP++Tat-βsyn-degron vs MPP+: ##P<0.01) and by the decreased numbers of TH-positive neurons (e; N=8; F(3,21)=38.00, P<0.001; Bonferroni post hoc test: MPP++Tat-βsyn-degron vs MPP+: ###P<0.001). d: representative TH-positive neuron staining. Data are presented as mean±S.E.M. The statistical difference between groups was determined by two-way ANOVA, followed by Bonferroni post hoc test. **P≤0.01 and ***P≤0.001 compared with the control. ##P≤0.01 and ###P≤0.001 indicate the statistical difference between the MPP++peptide group and the MPP+group. n.s. denotes not significant. Scale bar in d: 50 μm.

FIG. 5 illustrates a protective effect of Tat-βsyn-degron against α-synuclein propagation in a mouse model of synucleinopathy. (a) The experimental timeline. M83 mice were injected i.e with PBS or PFFs and i.p. with PBS, Tat-Rsyn peptide or Tat-βsyn-degron. B: behavioral tests. Vertical red lines: the days that the mice received i.p. injections. Vertical white lines: the days off i.p. injections. (b and d) Phosphorylated (pS129) α-synuclein immunostaining of coronal sections at the level of substantia nigra (b) and pons (d) from M83 mice. (c and e) Magnified images expanded from boxes in b or d showing pS129 α-synuclein staining. (f and g) Histogram showing the total pS129 α-synuclein (Total pSyn) staining per area at the level of substantia nigra (f, PBS: N=3; Tat-βsyn: N=6; Tat-βsyn-degron: N=7; t=2.235, df=11, P<0.05) and pons (g, PBS: N=3; Tat-βsyn: N=7; Tat-βsyn-degron: N=6; t=2.180, df=11, P=0.05) quantified from b and d. (h and j) Microglial Iba-1 immunostaining of coronal sections at the level of substantia nigra (h) and pons (j) from M83 mice. (i and k) Magnified images expanded from boxes in h and j showing Iba-1 staining. (I and m) Histogram showing the total Iba1 staining per area at the level of substantia nigra (I, PBS: N=2; Tat-βsyn: N=6; Tat-βsyn-degron: N=8; F(2,13)=1.61, P=0.238) and pons (m, PBS: N=3; Tat-βsyn: N=7; Tat-βsyn-degron: N=8; F(2,15)=5.14, P<0.05, Bonferroni post hoc test: Tat-βsyn-degron vs Tat-βsyn: P<0.05) quantified from h and l. Data are presented as mean±S.E.M. Statistical significance in f and g was determined by unpaired t test (because the PBS group did not have any pS129 α-synuclein staining and all the values were 0). Statistical significance in l and m was determined by one-way ANOVA, followed by Bonferroni post hoc test. n.s. denotes not significant. ml: medial lemniscus, PCG: pontine central grey, SNc: subtantia nigra pars compacta, v4: 4th ventricle, VTA: ventral tegmental area. Scale bar: 200 μm in b, d, h and j; 25 μm in c, e, i and k.

FIG. 6 illustrates a protective effect of Tat-βsyn-degron peptide-mediated knockdown of α-synuclein against parkinsonian toxin MPTP-induced TH protein decrease. (a-f) Mice received i.p. injections of MPTP (30 mg/kg) or same volumes of saline once a day for 5 days, along with Tat-βsyn-degron or its control Tat-βsyn (6 μmol/kg; i.p.) twice a day for 12 days. Brain tissues were collected for immunoblotting for α-synuclein and TH immediately after behavioral assessments on day 12. Tat-βsyn-degron, but not Tat-βsyn, significantly reduced α-synuclein in the substantia nigra-containing ventral midbrain (a and b; N=10; F(3,27)=5.28, P<0.01; Bonferroni post hoc test: MPTP+Tat-βsyn-degron vs MPTP: P=0.461) and the striatum (d and e; N=11; F(3,30)=7.24, P<0.01; Bonferroni post hoc test: MPTP+Tat-βsyn-degron vs MPTP: P=0.070), and protected against the MPTP-induced decrease in the level of TH protein in both ventral midbrain (a and c; N=10; F(3,27)=5.97, P<0.01; Bonferroni post hoc test: MPTP+Tat-βsyn-degron vs MPTP: P=0.349) and striatum (d and f; N=9; F(3,24)=8.33, P<0.01; Bonferroni post hoc test: MPTP+Tat-βsyn-degron vs MPTP: #P<0.05). Data are presented as mean±S.E.M. The statistical difference between groups was determined by two-way ANOVA, followed by Bonferroni post hoc test. *P≤0.05 and **P≤0.01 compared with the control. #P≤0.05 indicates the statistical difference between the MPTP+peptide group and the MPTP group. n.s. denotes not significant.

FIG. 7 illustrates a protective effect of Tat-βsyn-degron peptide-mediated knockdown of α-synuclein against parkinsonian toxin MPTP-induced dopaminergic neuronal damage and behavioral deficits in mice. (a-e) Mice received i.p. injections of MPTP (30 mg/kg) or same volumes of saline once a day for 5 days, along with Tat-βsyn-degron or its control Tat-βsyn (6 μmol/kg; i.p.) twice a day for 12 days. Brain tissues were collected for immunohistochemical staining of TH (a and c) immediately after behavioral assessments (e) on day 12. (a-d) Tat-βsyn-degron, but not its control Tat-βsyn, protected against MPTP-induced decrease in the number of dopaminergic neurons in the substantia nigra (SN) pars compacta as estimated by blinded neuron counting from bregma −2.92 mm to −3.64 mm (b; N=7 for the Tat-βsyn-degron group and N=8 for the other three groups; F(3,20)=7.02, P<0.01; Bonferroni post hoc test: MPTP+Tat-βsyn-degron vs MPTP: P=1.00) and the density of dopaminergic neuronal terminals in the striatum (ST; d; N=6 for all groups; F(3,15)=12.29, P<0.001; Bonferroni post hoc test: MPTP+Tat-βsyn-degron vs MPTP: #P<0.05). (e) Rotarod motor behavioral tests revealed that mice treated with chronic MPTP (MPTP; N=28) showed significantly shorter latency in falling off the rotarod compared with mice receiving saline control (Saline; N=28), and that the MPTP-induced motor deficits were significantly reduced by Tat-βsyn-degron (MPTP+Tat-βsyn-degron; N=28) and by Tat-βsyn (MPTP+Tat-βsyn; N=28) (F(3,108)=4.94; P<0.01; Bonferroni post hoc test: compared with the MPTP group: saline, **P<0.01; MPTP+Tat-βsyn: *P<0.05; MPTP+Tat-βsyn-degron: *P<0.05). All data are presented as mean±S.E.M. The statistical difference between groups was determined by two-way ANOVA, followed by Bonferroni post hoc test. *P≤0.05, **P≤0.01 and ***P≤0.001 compared with the control in b and d and with the MPTP group in e. #P≤0.05 indicates the statistical difference between the MPTP+peptide group and the MPTP group. n.s. denotes not significant. Scale bar: 0.5 mm in a and 1 mm in c.

### DETAILED DESCRIPTION

As used herein, the term “Tat-βsyn-degron” refers to the following amino acid sequence: YGRKKRRQRRRRTKSGVYLVGRRRG [SEQ ID NO: 1].

The present disclosure relates to a method of treating a disease characterized by abnormal aggregation of α-synuclein in a subject, the method comprising administering to the subject a therapeutically effective amount of a peptide comprising an α-synuclein binding domain operably linked to a protein transduction domain and a proteasomal targeting domain, wherein the α-synuclein binding domain is derived from a reversed sequence of β-synuclein.

The present disclosure also relates to a method of reducing neuroinflammation in a subject having a disease that is, or is characterized by, a synucleinopathy, the method comprising administering to the subject a therapeutically effective amount of a peptide comprising an α-synuclein binding domain operably linked to a protein transduction domain and a proteasomal targeting domain, wherein the α-synuclein binding domain is derived from a reversed sequence of β-synuclein.

The present disclosure also relates to a method of reducing the cell-to-cell propagation of α-synuclein in the brain of a subject having a disease that is, or is characterized by, a synucleinopathy, the method comprising administering to the subject a therapeutically effective amount of a peptide comprising an α-synuclein binding domain operably linked to a protein transduction domain and a proteasomal targeting domain, wherein the α-synuclein binding domain is derived from a reversed sequence of β-synuclein.

The present disclosure also relates to a use of a therapeutically effective amount of a peptide comprising an α-synuclein binding domain operably linked to a protein transduction domain and a proteasomal targeting domain, wherein the α-synuclein binding domain is derived from a reversed sequence of β-synuclein, for the treatment of a subject having a disease characterized by the abnormal aggregation of α-synuclein.

The present disclosure also relates to a use of a therapeutically effective amount of a peptide comprising an α-synuclein binding domain operably linked to a protein transduction domain and a proteasomal targeting domain, wherein the α-synuclein binding domain is derived from a reversed sequence of β-synuclein, to reduce the cell-to-cell propagation of α-synuclein in a subject having a disease that is, or is characterized by, a synucleinopathy.

The present disclosure also relates to a use of a therapeutically effective amount of a peptide comprising an α-synuclein binding domain operably linked to a protein transduction domain and a proteasomal targeting domain, wherein the α-synuclein binding domain is derived from a reversed sequence of β-synuclein, to reduce neuroinflammation in a subject having a disease that is, or is characterized by, a synucleinopathy.

The present disclosure also relates to a pharmaceutical composition for administration to a subject having a disease that is, or is characterized by, a synucleinopathy, the pharmaceutical composition comprising (a) a therapeutically effective amount of a peptide comprising an α-synuclein binding domain operably linked to a protein transduction domain and a proteasomal targeting domain, wherein the α-synuclein binding domain is derived from a reversed sequence of β-synuclein; and (b) a carrier.

Embodiments of these methods, uses, and compositions may include any one of or a combination of any two or more of the following features:
Abnormal aggregation of α-synuclein in one or more regions of the brain of the subject is reduced.
Inflammation in one or more regions of the brain of the subject is reduced.
One or more symptoms of the disease are ameliorated.
The binding affinity of the peptide for α-synuclein is significantly greater than the binding affinity of the peptide for: (i) β-synuclein; or (ii) γ-synuclein.
The α-synuclein binding domain comprises an amino acid sequence substantially identical to SEQ ID NO: 2.
The protein transduction domain comprises an amino acid sequence selected from the group consisting of:
   the HIV Tat transduction domain, YGRKKRRQRRR;
   the Drosophila melanogaster Antennapedia domain Antp (amino acids 43-58), RQIKWFQNRRMKWKK;
   Buforin II, TRSSRAGLQFPVGRVHRLLRK;
   hClock-(amino acids 35-47) (human Clock protein DNA-binding peptide), KRVSRNKSEKKRR;
   MAP (model amphipathic peptide), KLALKLALKALKAALKLA;
   K-FGF, AAVALLPAVLLALLAP;
   Ku70 derived peptide, comprising a peptide selected from the group comprising VPMLKE, VPMLK, PMLKE or PMLK;
   Prion, Mouse Prpe (amino acids 1-28), MANLGYWLLALFVTMWTDVGLCKKRPKP;
   pVEC, LLIILRRRIRKQAHAHSK;
   Pep-I, KETWWETWWTEWSQPKKKRKV;
   SynB1, RGGRLSYSRRRFSTSTGR;
   Transportan, GWTLNSAGYLLGKINLKALAALAKKIL;
   Transportan-10, AGYLLGKINLKALAALAKKIL;
   CADY, Ac-GLWRALWRLLRSLWRLLWRA-cysteamide;
   Pep-7, SDLWEMMMVSLACQY;
   FIN-1, TSPLNIHNGQKL;
   VT5, DPKGDPKGVTVTVTVTVTGKGDPKPD; or
   pISL, RVIRVWFQNKRCKDKK.
The proteasomal targeting domain comprises a degron, which may comprise the amino acid sequence RRRG.
The disease is selected from Parkinson's disease, diffuse Lewy body disease, transitional Lewy body dementia, and multiple system atrophy.
The subject is a human or other animal, such as a dog.
The administration of the peptide is by systemic administration, such as intravenous administration.
The peptide comprises an amino acid sequence having at least about 90% sequence identity to, or at least about 95% sequence identity to, or comprising, or consisting of, SEQ ID NO: 1.

Preferred embodiments will be described with reference to the following exemplary information which should not be used to limit or construe the invention.

Proteins can be degraded by targeting them for either lysosomal or proteasomal degradation in the cell; however, depending on pathological conditions, lysosomes, proteasomes, or both can become compromised. For example, several lysosome-related gene mutations have been linked to Parkinson's disease. In addition to LRRK2 G2019S (Orenstein et al., 2013), mutations in several other proteins have also been linked with lysosomal dysfunction in PD, such as ATP13A2 and ATP6AP2, two types of ATPases that are found on the lysosome membrane. Altered splicing of ATP6AP2 has been observed in X-linked parkinsonism (Korvatska et al., 2013; Gupta et al., 2015) and a variety of mutations in ATP13A2 have been linked with juvenile and early-onset recessively inherited parkinsonism (Trinh and Farrer, 2013). Therefore, when contemplating methods of treating PD and other synucleinopathies by reducing the pathological aggregation of α-synuclein, it may be beneficial to have a protein degradation system that can target the proteasome.

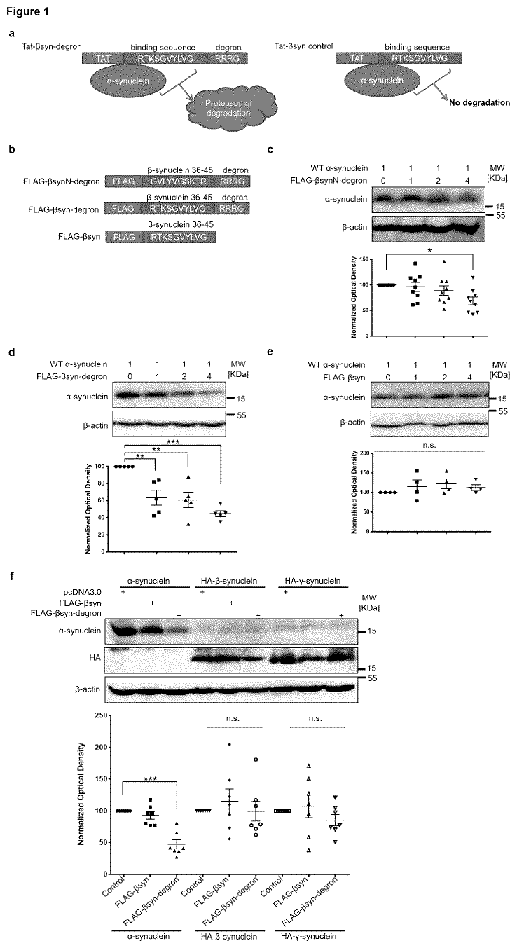

Referring to FIGS. 1a and 1b, one example of an α-synuclein targeting, proteasome-dependent degradation peptide is illustrated. The illustrated peptide is composed of three domains: 1) an α-synuclein-binding domain; 2) a protein transduction domain; and 3) a proteasomal targeting domain.

In some embodiments, the α-synuclein-binding domain is derived from β-synuclein. In the illustrated embodiment, the α-synuclein-binding domain is derived from a reversed sequence of amino acids 36-45 of β-synuclein (“βsyn”; SEQ ID NO: 2—RTKSGVYLVG), and can specifically bind to monomeric α-synuclein with high affinity. In other embodiments, the natural βsyn sequence is used (“βsynN”; SEQ ID NO: 3—GVLYVGSKTR).

The protein transduction domain can be any synthetic or naturally-occurring amino acid sequence that can mediate the introduction of proteins and peptides into a cell. In some embodiments, the protein transduction domain may be selected from among the examples provided at https://www.lifetein.com/Cell_Penetrating_Peptides.html. In some embodiments, the protein transduction domain may be selected from the group consisting of:
a. the HIV Tat transduction domain, YGRKKRRQRRR;
a. the Drosophila melanogaster Antennapedia domain Antp (amino acids 43-58), RQIKWFQNRRMKWKK;
b. Buforin II, TRSSRAGLQFPVGRVHRLLRK;
c. hClock-(amino acids 35-47) (human Clock protein DNA-binding peptide), KRVSRNKSEKKRR;
d. MAP (model amphipathic peptide), KLALKLALKALKAALKLA;
e. K-FGF, AAVALLPAVLLALLAP;
f. Ku70 derived peptide, comprising a peptide selected from the group comprising VPMLKE, VPMLK, PMLKE or PMLK;
g. Prion, Mouse Prpe (amino acids 1-28), MANLGYWLLALFVTMWTDVGLCKKRPKP;
h. pVEC, LLIILRRRIRKQAHAHSK;
i. Pep-I, KETWWETVWWTEWSQPKKKRKV;
j. SynB1, RGGRLSYSRRRFSTSTGR;
k. Transportan, GWTLNSAGYLLGKINLKALAALAKKIL;
l. Transportan-10, AGYLLGKINLKALAALAKKIL;
m. CADY, Ac-GLWRALWRLLRSLWRLLWRA-cysteamide;
n. Pep-7, SDLWEMMMVSLACQY;
o. FIN-1, TSPLNIHNGQKL;
p. VT5, DPKGDPKGVTVTVTVTVTGKGDPKPD; or
q. pISL, RVIRVWFQNKRCKDKK.

In the illustrated embodiment, the protein transduction domain is HIV Tat, which has been shown to be capable of delivering peptides across both the BBB and the plasma membrane of neurons following a systemic administration in freely moving animals and humans. Specifically, the protein transduction domain of the illustrated embodiment is the HIV Tat transduction domain, YGRKKRRQRRR.

The proteasomal targeting domain can be any peptide signal that can direct its tagged proteins to proteasomes for degradation. In the illustrated embodiment, the proteasomal targeting domain is a degron comprised of the amino acid sequence RRRG. In another embodiment, the proteasomal targeting domain is a degradation peptide derived from the N terminal of second mitochondria-derived activator of caspase (SMAC). The degradation peptide may comprise the amino acid sequence AVPIAQ, AVPI, or AVPIAQKS. In a preferred embodiment, the degradation peptide comprises the amino acid sequence AVPIAQ.

As used herein, ‘peptide’ or ‘polypeptide’ may be used interchangeably, and generally refer to a compound comprised of at least two amino acid residues covalently linked by peptide bonds or modified peptide bonds. Modified peptide bonds may include for example peptide isosteres (modified peptide bonds) that may provide additional desired properties to the peptide, such as increased half-life. The amino acids comprising a peptide or polypeptide described herein may also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification may be present in the same or varying degrees at several sites in a given peptide.

Amino acids are molecules containing an amine group, a carboxylic acid group and a side chain that varies between different amino acids. An amino acid may be in its natural form or it may be a synthetic amino acid. An amino acid may be described as, for example, polar, non-polar, acidic, basic, aromatic or neutral. A polar amino acid is an amino acid that may interact with water by hydrogen bonding at biological or near-neutral pH. The polarity of an amino acid is an indicator of the degree of hydrogen bonding at biological or near-neutral pH. Examples of polar amino acids include serine, proline, threonine, cysteine, asparagine, glutamine, lysine, histidine, arginine, aspartate, tyrosine and glutamate. Examples of non-polar amino acids include glycine, alanine, valine, leucine, isoleucine, methionine, phenylalanine, and tryptophan. Acidic amino acids have a net negative charge at a neutral pH. Examples of acidic amino acids include aspartate and glutamate. Basic amino acids have a net positive charge at a neutral pH. Examples of basic amino acids include arginine, lysine and histidine. Aromatic amino acids are generally nonpolar, and may participate in hydrophobic interactions. Examples of aromatic amino acids include phenylalanine, tyrosine and tryptophan. Tyrosine may also participate in hydrogen bonding through the hydroxyl group on the aromatic side chain. Neutral, aliphatic amino acids are generally nonpolar and hydrophobic. Examples of neutral amino acids include alanine, valine, leucine, isoleucine and methionine. An amino acid may be described by more than one descriptive category.

It will be appreciated by a person of skill in the art that aspects of the individual amino acids in a peptide described herein may be substituted. Furthermore, it will be appreciated by a person of skill in the art that certain substitutions are more likely to result in retention of activity. For example, amino acids sharing a common descriptive category may be substitutable for each other in a peptide.

Amino acids comprising the peptides described herein will be understood to be in the L- or D-configuration. Amino acids described herein may be modified by methylation, amidation, acetylation or substitution with other chemical groups which may change the circulating half-life of the peptide without adversely affecting their biological activity.

The term “identity” as used herein refers to the measure of the identity of sequence between two peptides. Identity can be determined by comparing a position in each sequence which may be aligned for purposes of comparison. For example, identity may be determined by the BLAST algorithm currently in use and which was originally described in Altschul et al. (1990) J. Mol. Biol. 215:403-410. The BLAST algorithm may be used with the published default settings. When a position in the compared sequence is occupied by the same amino acid, the molecules are considered to have shared identity at that position. The degree of identity between sequences is a function of the number of matching positions shared by the sequences and the degree of overlap between the sequences. Furthermore, when considering the degree of identity with SEQ ID NOs: 1, 2, or 3, it is intended that the equivalent number of amino acids be compared to SEQ ID NOs: 1, 2, or 3, respectively. Additional sequences (i.e. other than those corresponding to the 25 or 10 amino acids of SEQ ID NOs: 1 or SEQ ID Nos: 2 or 3, respectively), are not intended to be considered when determining the degree of identity with SEQ ID NOs: 1, 2, or 3. The sequence identity of a given sequence may be calculated over the length of the reference sequence (i.e. SEQ ID NOs: 1, 2, or 3).

Nomenclature used to describe the peptides or polypeptides may follow the conventional practice where the amino group is presented to the left and the carboxy group to the right of each amino acid residue. In the sequences representing selected specific embodiments of the present invention, the amino- and carboxy-terminal groups, although not specifically shown, will be understood to be in the form they would assume at physiologic pH values, unless otherwise specified. In the amino acid structure formulae, each residue may be generally represented by a one-letter or three-letter designation, corresponding to the name of the amino acid, in accordance with the following Table A.

TABLE A
Nomenclature and abbreviations of the 20 standard L-amino
acids commonly found in naturally occurring peptides

| Full name | Three-letter abbreviation | One-letter abbreviation |
|---|---|---|
| Alanine | Ala | A |
| Cysteine | Cys | C |
| Aspartic acid | Asp | D |
| Glutamic acid | Glu | E |
| Phenylalanine | Phe | F |
| Glycine | Gly | G |
| Histidine | His | H |
| Isoleucine | Ile | I |
| Lysine | Lys | K |
| Leucine | Leu | L |
| Methionine | Met | M |
| Asparagine | Asp | N |
| Proline | Pro | P |
| Glutamine | Gln | Q |
| Arginine | Arg | R |
| Serine | Ser | S |
| Threonine | Thr | T |
| Valine | Val | V |
| Tryptophan | Trp | W |
| Tyrosine | Tyr | Y |

One or both, but usually one terminus of the peptide, may be substituted with a lipophilic group, usually aliphatic or aralkyl group, which may include heteroatoms. Chains may be saturated or unsaturated. Conveniently, commercially available aliphatic fatty acids, alcohols and amines may be used, such as caprylic acid, capric acid, lauric acid, myristic acid and myristyl alcohol, palmitic acid, palmitoleic acid, stearic acid and stearyl amine, oleic acid, linoleic acid, docosahexaenoic acid, etc. Preferred are unbranched, naturally occurring fatty acids between 14-22 carbon atoms in length. Other lipophilic molecules include glyceryl lipids and sterols, such as cholesterol. The lipophilic groups may be reacted with the appropriate functional group on the peptide in accordance with conventional methods, frequently during the synthesis on a support, depending on the site of attachment of the oligopeptide to the support. Lipid attachment is useful, for example, where peptides may be introduced into the lumen of a liposome, optionally along with other therapeutic agents, for administering the peptides and optionally agents into a host.

Depending upon their intended use, particularly for administration to mammalian hosts, the subject peptides may also be modified by attachment to other compounds for the purposes of incorporation into carrier molecules, changing peptide bioavailability, extending or shortening half-life, controlling distribution to various tissues or the blood stream, diminishing or enhancing binding to blood components, and the like.

The peptides herein may comprise a delivery and targeting (dat) moiety. The term delivery and targeting (dat) moiety as used herein is meant to encompass any moiety that assists in delivering and/or targeting the peptides described herein to a target cell or tissue or within a target cell or within the cells of a target tissue. For example, the dat moiety may be a cell membrane penetrating sequence. Furthermore, a dat moiety may “assist” in delivery and/or targeting by virtue of promoting the biological efficacy of the peptides described herein. Moieties that enable delivery or targeting of bioactive molecules into cells in a suitable manner so as to provide an effective amount, such as a pharmacologically effective amount, are known in the art. Optionally, the delivery and targeting (dat) moiety may comprise, or may be selected from, one or more of: receptor ligands, protein transduction domains, micelles, liposomes, lipid particles, viral vectors, peptide carriers, protein fragments, or antibodies. Optionally, the protein transduction domain may be the cell-membrane transduction domain of HIV-1 Tat (Demarchi et al. (1996) J Virol. 70: 4427-4437). Other examples and related details of such protein transduction domains are described and known to those skilled in the art.

In therapeutic applications, the compositions described herein may be administered to a subject having a disease or condition, such as (but not limited to) a synucleinopathy, which may be PD, DLB, or MSA. The composition described herein may be administered to a subject in an amount sufficient to cure or at least partially arrest or reduce at least one manifestation of the disease or condition and/or its complications or to help alleviate at least one symptom associated therewith. Such an amount is defined as a “therapeutically effective amount” or an “effective amount”. Amounts effective for this use will depend upon the severity of the disease or condition, the intended use (treatment, cure, prophylactic, alleviation of symptoms, etc.) and the general state of the subject's health. Single or multiple administrations of the compositions may be administered depending on the dosage and frequency as required and tolerated by the patient. A composition generally would provide a sufficient quantity of the active peptide or peptides described herein to effectively treat (for example, to at least ameliorate one or more symptoms) in the subject.

The concentration(s) of peptide described herein can vary widely, and may be selected primarily based on fluid volumes, viscosities, body weight and the like in accordance with the particular mode of administration selected and the subject's needs. Concentrations, however, will typically be selected to provide dosages ranging from about 0.01 or 1 mg/kg/day to about 50 mg/kg/day and sometimes higher. It will be appreciated that such dosages may be varied to optimize a therapeutic regimen in a particular subject or group of subjects.

Additional active therapeutic ingredients may be administered to the subject along with or prior to the primary active agent, e.g., the exemplary peptides described herein. The exemplary peptide may be co-administered with one or more other therapeutically active agents to enhance the therapeutic effect on the target cell or tissue by delivering another compound or compounds with a similar or complementary activity.

Peptides may be prepared in a number of ways. Chemical synthesis of peptides is well known in the art. Solid phase synthesis is commonly used and various commercial synthetic apparatuses are available, for example automated synthesizers by Applied Biosystems Inc., Foster City, Calif.; Beckman; etc. Solution phase synthetic methods may also be used, particularly for large-scale productions. Recombinant DNA, genetic and molecular engineering techniques are also known in the art.

Alternatively, or in addition, peptides may be generated in vivo via the delivery of an effective amount of an appropriate nucleic acid vector, such as a modified mRNA or DNA vector, to a subject. In one embodiment, a peptide may be generated via a nucleic acid vector comprising the sequence of bases 33-74 of SEQ ID NO: 4 (GGGGTGCTGTACGTGGGGAGCAAGACGAGGCGACGACGAGGC; SEQ ID NO: 8), or a sequence complementary or degenerate thereto, or a corresponding RNA sequence. In another embodiment, a peptide may be generated via a nucleic acid vector comprising the sequence of bases 37-78 of SEQ ID NO: 6 (CGTACTAAATCTGGTGTTTATTTGGTTGGTCGACGACGAGGC; SEQ ID NO: 9), or a sequence complementary or degenerate thereto, or a corresponding RNA sequence.

Peptides may also be provided in the form of a salt, generally in a salt form which is pharmaceutically acceptable. These include inorganic salts of sodium, potassium, lithium, ammonium, calcium, magnesium, iron, zinc, copper, manganese, and the like. Various organic salts of the peptide may also be made with, including, but not limited to, acetic acid, propionic acid, pyruvic acid, maleic acid, succinic acid, tartaric acid, citric acid, benzoic acid, cinnamic acid, salicylic acid, etc.

### Development of the Anti-Synucleinopathy Peptide, Tat-βsyn-Degron

As illustrated in FIG. 1b, to determine the efficacy and specificity of the sequence fragment as the binding domain of the present α-synuclein targeting peptide, two FLAG-tagged targeting peptide mini-genes (FLAG-βsynN-degron and FLAG-βsyn-degron), that encoded either natural or reverse amino acid sequences between 36-45 of β-synuclein, along with a degron targeting signal were constructed. A control mini-gene encoding FLAG-βsyn without degron was also constructed. HEK 293 cells were co-transfected with a human α-synuclein plasmid and one of these mini-genes.

As illustrated in FIG. 1e, co-transfection of FLAG-βsynN-degron or FLAG-βsyn-degron (FIGS. 1c and 1d), but not the control FLAG-βsyn peptide, resulted in a robust reduction in recombinant α-synuclein levels in a dose-dependent manner. While not wishing to be bound by any particular theory or mode of action, this may suggest that the targeting peptides, when co-expressed with α-synuclein, are sufficient to bind to α-synuclein and target it for proteasomal degradation.

Furthermore, FLAG-βsyn-degron appears to have had a better α-synuclein knockdown efficacy in comparison with FLAG-βsynN-degron (FIGS. 1c and 1d). Again, not while not wishing to be bound by any particular theory or mode of action, this enhanced α-synuclein knockdown efficacy is possibly due to an enhanced stability of the βsyn-degron peptide as compared to the βsynN-degron peptide.

The FLAG-βsyn-degron induced knockdown is believed to be target-specific, as this knockdown was not associated with a detectable change in β-actin levels (FIGS. 1c and 1d) and it was selective to α-synuclein, but not β- or γ-synuclein, the two other members of the synuclein protein family (FIG. 1f).

The efficacy of the present peptide to decrease endogenous α-synuclein in neurons in situ, using chemically synthesized, membrane-permeant α-synuclein targeting peptide Tat-βsyn-degron, along with a control Tat-βsyn peptide (as illustrated in FIG. 1a) was assessed.

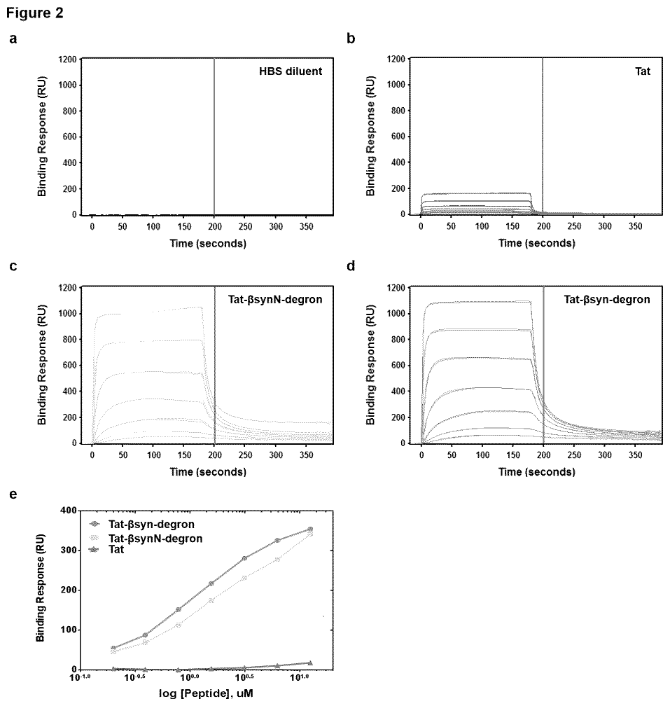

FIG. 2 illustrates results from Biacore peptide-protein binding assays, which confirmed that the illustrated example of the Tat-βsyn-degron robustly bound to purified recombinant α-synuclein.

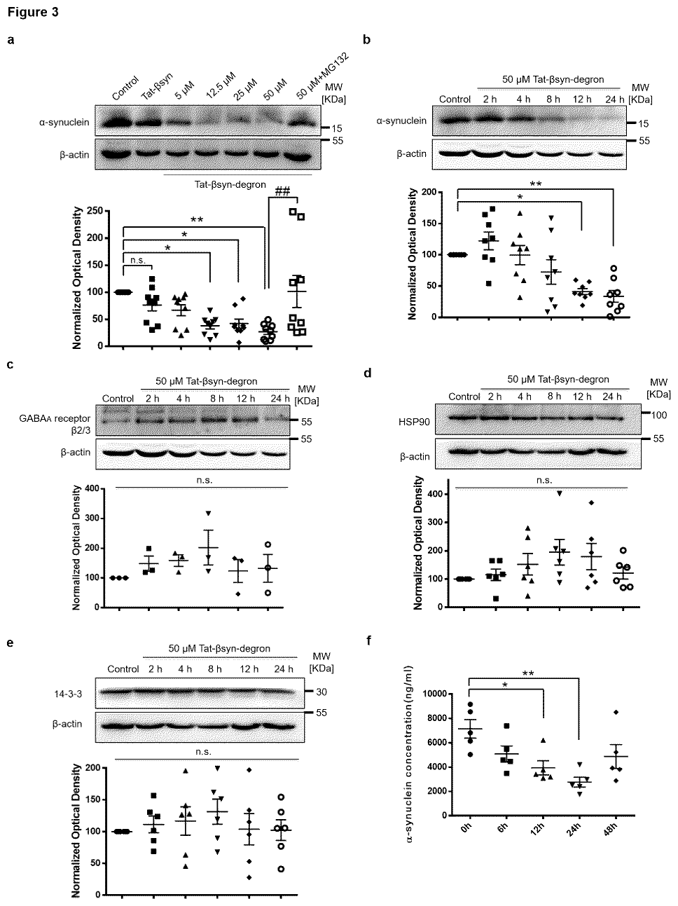

As illustrated in FIG. 3a, bath application of Tat-βsyn-degron, but not the control Tat-βsyn, for 24 hrs produced a dose-dependent reduction of endogenous α-synuclein in cortical neuron cultures. The proteasomal inhibitor MG132 (10 μM; 24 hrs) prevented Tat-βsyn-degron induced knockdown of α-synuclein, demonstrating that such knockdown is likely mediated by proteasomal degradation. Additionally, the knockdown was time dependent, and the α-synuclein level remained low at 24 hrs (FIG. 3b). The peptide-mediated knockdown was specific to α-synuclein, as it did not affect the levels of several other neuronal proteins surveyed in the same treated cultures, including transmembrane protein GABA_{A} receptor β2/3 subunit, intracellular protein HSP 90, and 14-3-3, a known α-synuclein binding protein (FIGS. 3c-e).

Tat-βsyn-degron peptide was also shown to decrease α-synuclein levels in vivo, using M83 transgenic mice that overexpress mutant human A53T α-synuclein. M83 mice, injected intra-peritoneally (i.p.) with 40 mg/kg Tat-βsyn-degron peptide, were sacrificed at time points of 6, 12, 24, and 48 hrs and α-synuclein levels in the brain were measured by ELISA. The Tat-βsyn-degron peptide led to a reduction of α-synuclein levels at both 12 hrs and 24 hrs, but not at 48 hrs, indicating that the effect of the Tat-βsyn-degron peptide in vivo is transient and peaks around 24 hrs in this mouse line (FIG. 3f).

### Evaluation of Tat-βsyn-Degron Peptide Against Parkinsonian Toxin Induced Neuronal Damage In Vitro

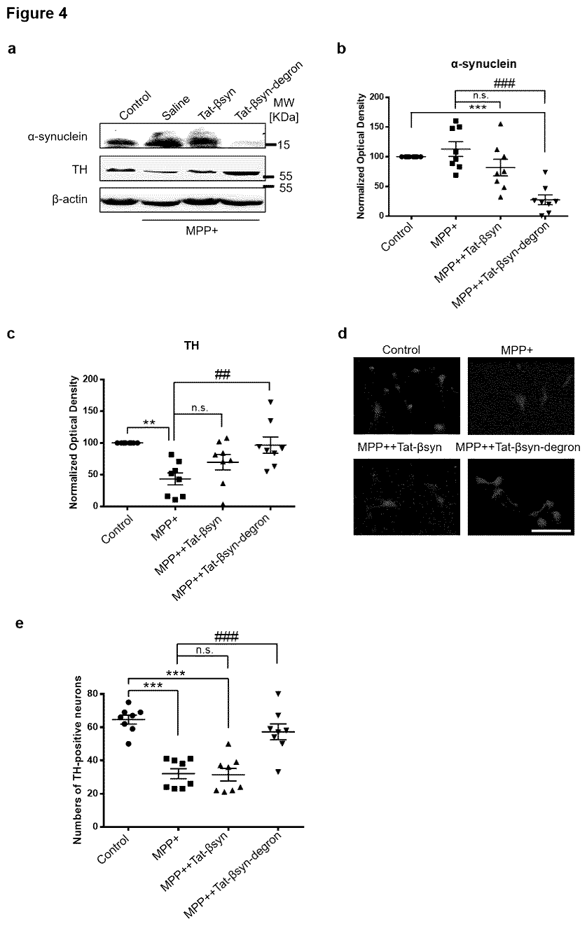

FIG. 4 illustrates data demonstrating the ability of the present peptide to protect dopaminergic neurons against MPP+ toxicity in vitro. As shown in FIG. 4, MPP+ treatment (20 μM; 48 hrs) induced the death of dopaminergic neurons in rat primary cultures of the ventral midbrain. This was demonstrated by the significant decrease in the level of tyrosine hydroxylase (TH), a dopaminergic neuronal marker protein (FIGS. 4a and 4c), and in the numbers of TH-positive neurons (FIGS. 4d and 4e). Bath application of the Tat-βsyn-degron peptide (25 μM; 48 hrs), but not the control Tat-βsyn peptide (25 μM; 48 hrs), induced a robust reduction in endogenous α-synuclein protein levels (FIGS. 4a and 4b). The reduction of α-synuclein almost fully protected dopaminergic neurons from MPP+ induced neurotoxicity, as shown by the rescue of TH protein levels (FIGS. 4a and 4c) and TH-positive neurons (FIGS. 4d and 4e) in the culture dishes.

### In Vivo Evaluation of the Tat-βsyn-Degron Peptide in a Mouse Model of Spreading Synucleinopathy

Accumulating evidence supports the role of prion-like propagation of α-synuclein in the pathogenesis of PD. To study whether the Tat-βsyn-degron peptide induced knockdown can reduce the propagation of α-synuclein in the brain, a mouse model of spreading synucleinopathy was established as described previously.

This model exhibits characteristic phenotypic features of synucleinopathies like PD, including increased α-synuclein aggregation and inflammation in defined regions of the brain, which are believed to have direct clinical relevance. As such, rescue of α-synuclein aggregation and inflammation in key brain loci in this model may significantly predict therapeutic utility.

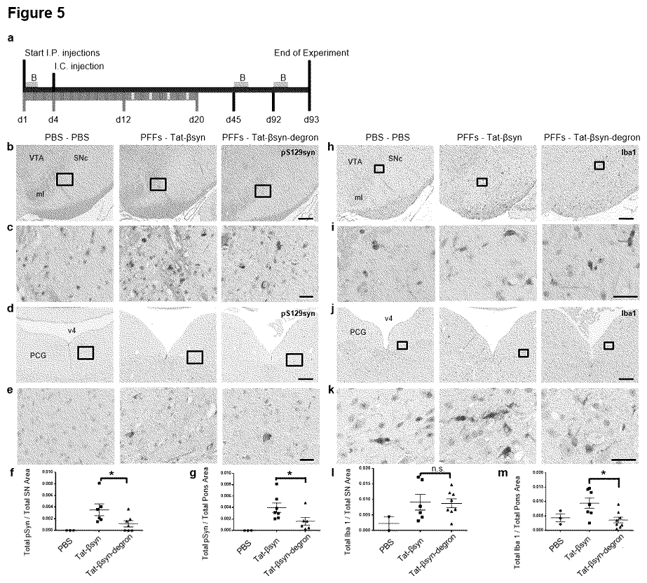

M83 mice were injected intracerebrally (i.e.) into the right dorsal striatum with either 12.5 μg of α-synuclein pre-formed fibrils (PFFs) or PBS. Starting at 3 days prior to the PFF injection, mice were treated daily for 12 days with either Tat-βsyn or Tat-βsyn-degron peptide (40 mg/kg; i.p.) and once every other day for the subsequent 8 days (20 days in total, FIG. 5a). At the end of three months, coronal brain sections of the M83 mice were prepared and stained with antibodies against serine 129-phosphorylated α-synuclein (pS129syn), a marker of pathogenic synuclein aggregates, and Iba-1, a calcium-binding protein specifically expressed in macrophage and microglia as a marker of neuroinflammation.

PFF, not PBS, inoculated animals exhibited some characteristic phenotypes of PD pathology, including increased α-synuclein aggregation and inflammation in defined regions of the brain, including the substantia nigra pars compacta and the pons (FIG. 5b-m). Mice injected with α-synuclein PFFs exhibited increased intensity of pS129syn staining (FIG. 5b-e). In these PFF-injected mice, pS129syn immunoreactive profiles were found in cell somas, dendritic branches and axons (FIGS. 5c and 5e). In comparison with the Tat-βsyn group, pS129syn staining was significantly reduced in both substantia nigra pars compacta (FIG. 5f) and pons (FIG. 5g) of the Tat-βsyn-degron treated group, indicating that the Tat-βsyn-degron peptide reduced propagation and seeding of α-synuclein aggregates in the brains of these mice.

The staining for Iba-1 could be detected throughout the brain in all mice (FIG. 5h-k). In comparison with the Tat-βsyn-treated group, the administration of Tat-βsyn-degron, while having no observable effect in the substantia nigra pars compacta (FIG. 5l), significantly reduced the level of Iba-1 staining in the pons region (FIG. 5m). Without wishing to be bound by any particular theory, these results may suggest that i.e. inoculation of α-synuclein PFFs produces inflammatory responses and that the inflammatory responses can be reduced in a region-specific manner by knocking down α-synuclein with the Tat-βsyn-degron peptide.

### In Vivo Evaluation of the Tat-βsyn-Degron Peptide in the MPTP Mouse Model of PD

While the aforementioned PFF-inoculation PD model demonstrates that the Tat-βsyn-degron peptide can reduce PFF-induced PD pathology, obvious PD-related behavioral phenotypes were not observed in either group of mice when behavioral tests were performed 45 days and three months after PFF injection. This lack of behavioral alterations, at the time points tested, precluded direct evaluation of the efficacy of peptide-induced α-synuclein knockdown in reducing PD behavioral phenotypes. Accordingly, to better evaluate the clinical relevance of the foregoing results, the therapeutic efficacy of the Tat-βsyn-degron peptide was further assessed in a pathologically and behaviorally well-characterized mouse dopaminergic neuron toxicity model of PD.

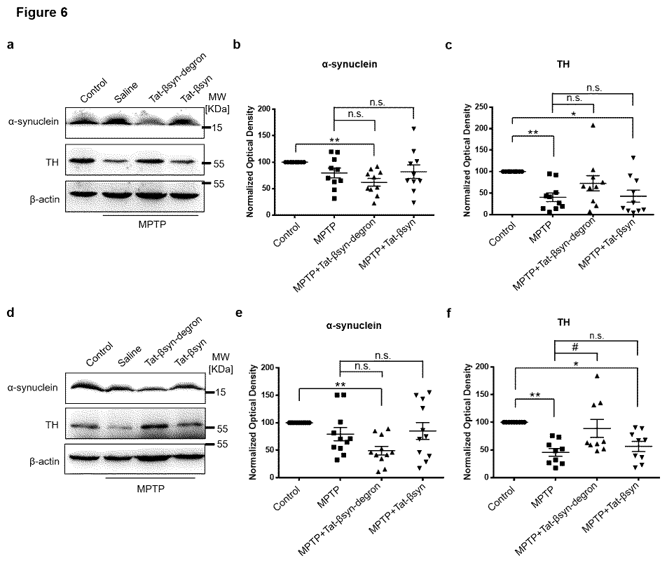

C57BL/6 mice were i.p. injected with 30 mg/kg parkinsonian toxin MPTP (or saline as control), once per day for 5 consecutive days, to induce dopaminergic neuron damage. The effects of MPTP administration on mouse rotarod performance and damage to dopaminergic neurons were then analyzed 1 week after the last injection of MPTP. To determine the effect of α-synuclein knockdown in protecting dopaminergic neurons against MPTP, the Tat-βsyn-degron peptide or its control Tat-βsyn (6 μmol/kg; i.p.) was used in some animals twice a day for 12 days, beginning the first day of MPTP injection. As shown in FIG. 6, the Tat-βsyn-degron peptide, but not Tat-βsyn peptide, induced significant α-synuclein degradation in both substantia nigra-containing ventral midbrain (FIGS. 6a and 6b) and striatum (FIGS. 6d and 6e). The striatum is a region that receives dopaminergic neuronal projections from the substantia nigra and is also deeply affected in PD. Consistent with the specific neurotoxic effects of MPTP on dopaminergic neurons, in mice receiving MPTP injection alone there was a significant loss of protein TH in both dopaminergic neurons in the substantia nigra-containing ventral midbrain (FIGS. 6a and 6c) and dopaminergic neuronal terminals in the striatum (FIGS. 6d and 6f). This was demonstrated by quantitative immunoblotting analysis of TH protein levels. As expected, the specific knockdown of α-synuclein by Tat-βsyn-degron protected against MPTP-induced dopaminergic neuronal injury (FIGS. 6c and 6f).

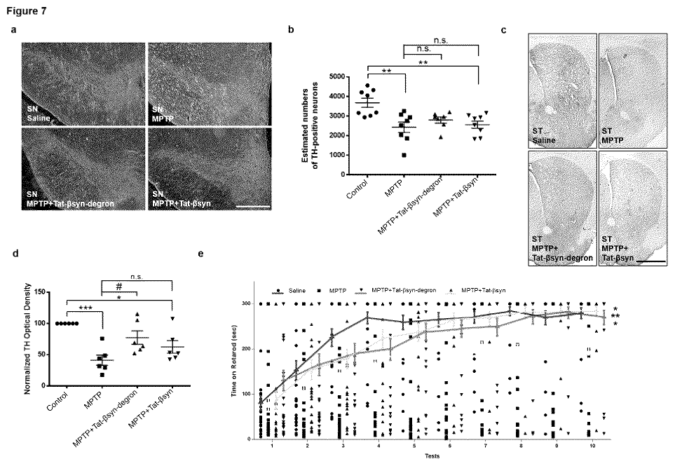

The neuroprotective effects of Tat-βsyn-degron peptide were further supported by immunohistochemical analysis. In mice receiving only MPTP injections, there was a significant loss of TH-positive neurons in the substantia nigra pars compacta as estimated by blinded neuron counting from bregma −2.92 mm to −3.64 mm (FIGS. 7a and 7b) and TH-positive neuronal terminals in the striatum as quantified with densitometric analysis (FIGS. 7c and 7d). As expected, the MPTP-induced dopaminergic neuronal damage was largely protected by the Tat-βsyn-degron peptide, but not the Tat-βsyn peptide (FIG. 7a-d).

The motor function of these mice was tested by a rotarod test using a protocol modified from a previous study. Consistent with the dramatic effects of α-synuclein knockdown and its protection of dopaminergic neurons from MPTP-induced neurotoxicity, the Tat-βsyn-degron peptide also significantly rescued the MPTP-induced motor deficits in a rotarod behavioral test (FIG. 7e). Interestingly, treatment with Tat-βsyn, while producing a small, non-significant decrease in MPTP-induced neuronal damage (FIG. 6 and FIG. 7a-d), also reduced motor deficits in mice (FIG. 7e). Without wishing to be bound by any particular theory, it is believed that this effect may be in part due to the possibility that Tat-βsyn can function as an interference peptide to inhibit α-synuclein oligomerization. Consistent with this conjecture, homology alignment analysis shows that the amino acids 36-45 of β-synuclein (βsyn) is quite similar to a sequence found in the N-terminal lipid-binding domain of α-synuclein, a domain that may be involved in the self-oligomerization among α-synucleins. The Tat-βsyn-degron peptide (6 μmol/kg, i.p.) was also able to similarly and significantly reduce the α-synuclein expression in the kidney, the spleen, the ventral midbrain and the striatum 6 hours after peptide injection, indicating that the knockdown of endogenous α-synuclein by the Tat-βsyn-degron peptide is not limited to the central nervous system.

### Protective Effect of Tat-βSyn-Degron Peptide Against Parkinsonian Toxin Induced Neuronal Damage In Vivo

FIG. 6 illustrates use of the Tat-βsyn-degron peptide in a mouse parkinsonian toxicity model of PD. C57BL/6 mice were i.p. injected with 30 mg/kg parkinsonian toxin MPTP (or saline as control), once per day for 5 consecutive days, to induce dopaminergic neuron damage. The effects of MPTP administration on mouse rotarod performance and damage to dopaminergic neurons were then analyzed 1 week after the last injection of MPTP. To determine the effect of α-synuclein knockdown in protecting dopaminergic neurons against MPTP, Tat-βsyn-degron peptide or its control Tat-βsyn (6 μmol/kg; i.p.) was used in some animals twice a day for 12 days, beginning the first day of MPTP injection.

As illustrated in FIG. 7, the Tat-βsyn-degron peptide, but not the Tat-βsyn peptide, induced significant α-synuclein degradation in both substantia nigra-containing ventral midbrain (FIGS. 7a and 7b) and striatum (FIGS. 7d and 7e). The striatum is a region that receives dopaminergic neuronal projections from the substantia nigra and is also deeply affected in PD. Consistent with the specific neurotoxic effects of MPTP on dopaminergic neurons, in mice receiving MPTP alone, there was a significant loss of protein TH in both dopaminergic neurons in the substantia nigra-containing ventral midbrain (FIGS. 7a and 7c) and dopaminergic neuronal terminals in the striatum (FIGS. 7d and 7f). This was demonstrated by quantitative immunoblotting analysis of TH protein levels. The specific knockdown of α-synuclein by Tat-βsyn-degron protected against MPTP-induced dopaminergic neuronal injury (FIGS. 7c and 7f).

FIGS. 7a-d illustrate immunohistochemical analysis of the potential neuroprotective effects of the present Tat-βsyn-degron peptide. In mice receiving only MPTP injections, there was a significant loss of TH-positive neurons in the substantia nigra pars compacta as revealed by blinded neuron counting (FIGS. 7a and 7b) and TH-positive neuronal terminals in the striatum as quantified with densitometric analysis (FIGS. 7c and 7d). The MPTP-induced dopaminergic neuronal damage was largely protected by the Tat-βsyn-degron peptide, but not the Tat-βsyn peptide (FIG. 7a-d).

FIG. 7e illustrates the motor function of these mice, as tested by the rotarod test using a protocol modified from a previous study. Consistent with the effects of α-synuclein knockdown and its protection of dopaminergic neurons from MPTP-induced neurotoxicity, the rotarod behavioral test revealed that the Tat-βsyn-degron peptide also significantly rescued the MPTP-induced motor deficits (FIG. 7e). Treatment with Tat-βsyn, while producing a small, non-significant decrease in MPTP-induced neuronal damage (FIG. 6 and FIG. 7a-d) also reduced motor deficits in mice (FIG. 7e).

### DISCUSSION

The present inventors have developed a peptide-based method having potential clinical application in the treatment of synucleinopathies such as PD, DLB and MSA. The peptide can rapidly and reversibly decrease the level of α-synuclein via proteasomal degradation, as compared to other known methods. By knocking down α-synuclein, and particularly abnormally aggregated α-synuclein, a major disease-causing molecule, the present peptide directly targets a disease-causing process and may stop or slow down the progression of disease, as compared to other known therapeutic strategies for synucleinopathies, which do not directly target the disease-causing processes, instead being related to symptom-relieving (e.g., deep brain stimulation and many pharmacological treatments). The present method also contemplates the simpler and more effective delivery of the present BBB- and membrane-permeable peptide into neurons in the brain following a non-invasive systemic administration, as compared to other known techniques.

The present method employing a Tat-βsyn-degron peptide may efficiently decrease levels of α-synuclein with high specificity, thereby protecting dopaminergic neurons from MPP+induced neurotoxicity in a cell culture model of Parkinson's disease. Furthermore, the Tat-βsyn-degron peptide may cross the BBB and enter dopaminergic neurons in the brain to knock down endogenous α-synuclein, as well as reduce MPTP-induced neuronal death in the substantia nigra and behavioral deficits following intraperitoneal administration in a mouse MPTP toxicity model of PD. In addition, systemic administration in a mouse PFF model of PD indicates that the Tat-βsyn-degron peptide may prevent both α-synuclein aggregation and inflammation in specific brain areas, thus targeting characteristic phenotypes of synucleinopathies such as PD. As such, the present Tat-βsyn-degron peptide may represent a disease-modifying anti-synucleinopathy therapeutic.

By knocking down aggregated α-synuclein, one of the major disease-causing molecules, the present method employs Tat-βsyn-degron to directly target one of the disease-causing processes and may be expected to stop or slow down the progression of the associated disease. This approach contrasts with many of the anti-synucleinopathy strategies currently used in the clinic. For example, deep brain stimulation and currently available pharmacological treatments are not believed to directly target the disease-causing processes, and therefore may be symptom-relieving but may not stop or slow down the progression of the disease.

The present method also may have advantages over other protein-knockdown technologies, such as anti-sense and siRNA. For example, although siRNA-mediated knockdown of α-synuclein has been shown to be effective in various models of PD, the clinical applications can be hindered by an inability to cross the BBB and the plasma membrane of neurons. The delivery of siRNAs to the brain is mainly accomplished by an invasive intracerebral injection or viral infection, which may not be clinically practical for the therapeutic use in human patients. Several recent studies suggest that delivery of siRNA to the brain by a non-invasive systemic injection may be achieved by coupling siRNA with brain delivery vehicles such as RVG-9R peptide or RVG-9R peptide-coated exosome. However, these techniques may be restricted to the acetylcholine receptor-expressing neurons in the brain or remain technically challenging. The present peptide-based method may be simpler and more effectively delivered into neurons in the brain following a non-invasive systemic administration. The effectiveness is demonstrated by the high efficacy of the peptide in knocking down α-synuclein in the brain and robust neuroprotective efficacy in two different animal models of PD (FIGS. 5, 6 and 7).

Additionally, the present peptide-mediated knockdown may have a temporal advantage over antisense or siRNA-mediated knockdown. α-synuclein is a stable protein with a long half-life and it may take a few weeks for siRNA to induce a significant reduction of endogenous α-synuclein protein level in the brain, whereas by hijacking the endogenous proteasomal degradation system in the cell, the present peptide may produce a rapid and robust degradation of α-synuclein protein within a few hours (FIGS. 3b and 3f).

The Tat-βsyn-degron peptide was demonstrated to reduce α-synuclein levels in a transgenic mouse model (M83) overexpressing human mutant A53T α-synuclein (FIG. 3f). Using PFF injections into the striatum of this mouse line, the Tat-βsyn-degron peptide was also shown to reduce the cell-to-cell propagation of pathology, as manifested by a reduction in pS129syn staining and Iba-1 microglial neuroinflammatory infiltration, two well-established hallmarks of synucleinopathy. This PFF-injection mouse model is increasingly recognized as a robust system reconstituting the prion-like propagation hypothesis of pathogenesis, including in synucleinopathies such as PD, DLB and MSA. Accordingly, these results support the clinical relevance of the present methods employing the Tat-βsyn-degron peptide.

### Experimental Examples

Embodiments will now be illustrated with reference to the following examples which should not be used to construe or limit the scope of the present disclosure.

### Animals

Male C57BL/6 mice (20-25 g, purchased from Charles River (Beijing Office, China)) were housed in plastic cages with free access to food and water and maintained in a temperature-controlled room (22° C.) with a 12/12 hrs light/dark cycle. All experimental protocols were approved by the Chongqing Medical University Animal Care Committee, and the methods were carried out in accordance with the approved guidelines and regulations. All efforts were made to minimize animal suffering and to reduce the number of animals used.

Male M83 hemizygous mice overexpressing the human A53T α-synuclein mutant (12 weeks old, 25-30 g, 004479, The Jackson Laboratory) were housed individually and maintained on a 12/12 hrs light/dark cycle at 22° C. ambient temperature and with unlimited access to food and water. Housing, breeding, and procedures were performed according to the Canadian Council on Animal Care and were approved by the McGill University Animal Care Committee.

### Chemicals and Reagents

Anti-α-synuclein antibody (BD Transduction Laboratories, 610786), anti-phosphorylated pS129 α-synuclein antibody (ab184674, Abcam), anti-β-actin antibody (Abcam, ab8227), anti-HA antibody (Roche, 118674231001), anti-tyrosine hydroxylase (TH) antibodies (BD Transduction Laboratories, 612300, for immunoblotting and immunohistochemistry; Novus, NB300-109, for immunocytochemistry), anti-Iba1 antibody (019-19741, Wako), anti-GABA_{A} receptor β2/3 antibody (Millipore, 05-474), anti-HSP90 antibody (BD Transduction Laboratories, 610418), anti-14-3-3 antibody (Millipore, 06-511), MG132 (Sigma, C2211), 1-Methyl-4-phenylpyridinium (MPP+) iodide (Sigma, D048), 1-Methyl-4-phenyl-1,2,3,6-tetrahydropyridine (MPTP) hydrochloride (Sigma, M0896), normal goat serum (G9023, Sigma Aldrich), goat anti rabbit horseradish peroxidase (111-035-144, Jackson ImmunoResearch), goat anti mouse horseradish peroxidase (115-035-146, Jackson ImmunoResearch). Tat-βsyn-degron peptide (YGRKKRRQRRRRTKSGVYLVGRRRG), Tat-βsynN-degron peptide (YGRKKRRQRRRGVLYVGSKTRRRRG) and Tat-βsyn control peptide (YGRKKRRQRRRRTKSGVYLVG) were chemically synthesized by GL Biochem (Shanghai, China). Tat peptide (YGRKKRRQRRR) was synthesized in our lab using the Prelude peptide synthesizer (Protein Technologies Inc.).

### Buffers and Media

Phosphate buffered saline (PBS, pH 7.4) contained 137 mM NaCl, 2.7 mM KCl, 8.1 mM Na₂HPO₄, and 1.76 mM KH₂PO₄. 1× Tris buffered saline containing 0.1% Tween-20 (TBST) pH 7.6 contained 20 mM trizma base, 150 mM sodium chloride and 0.1% Tween-20. Citrate buffer pH 6.0 contained 10 mM tri-sodium citrate. Cell lysis buffer contained 0.5% Triton X-100, 0.5% deoxycholic acid, and 1× protease and phosphatase inhibitor cocktail (Thermo Scientific, 78442) in sterile PBS. The 4× sample buffer contained 50% Glycerol, 125 mM pH 6.8 Tris-HCl, 4% SDS, 0.08% bromophenol blue, and 5% β-mercaptoethanol. Neuron culture media contained 2% B-27 supplement (Invitrogen, 17504-044) and 0.5 mM GlutaMax supplement (Invitrogen, 35050-061) in Neurobasal Media (Invitrogen, 21103-049).

### Plasmid Construction

The human α-synuclein plasmid was a generous gift from Dr. Hong Qing from Beijing Institute of Technology, China. The FLAG-βsynN-degron and FLAG-βsyn-degron peptide sequences were translated back to cDNA sequences and the corresponding sense and antisense DNA oligonucleotide strands were synthesized by Integrated DNA Technologies (IDT).

FLAG-βsynN-degron:
sense:
(SEQ ID NO: 4)
5′-AGCTTATGGACTACAAGGACGACGATGACAAGGGGGTGCTGTACGT
GGGGAGCAAGACGAGGCGACGACGAGGCTAAGC-3′
antisense:
(SEQ ID NO: 5)
5′-GGCCGCTTAGCCTCGTCGTCGCCTCGTCTTGCTCCCCACGTACAGC
ACCCCCTTGTCATCGTCGTCCTTGTAGTCCATA-3′
FLAG-βsyn-degron:
sense:
(SEQ ID NO: 6)
5′-CCCAAGCTTATGGACTACAAGGACGACGATGACAAGCGTACTAAAT
CTGGTGTTTATTTGGTTGGTCGACGACGAGGCTAAGCGGCCGCTTTTTT
CCTT-3′
antisense:
(SEQ ID NO: 7)
5′-AAGGAAAAAAGCGGCCGCTTAGCCTCGTCGTCGACCAACCAAATAA
ACACCAGATTTAGTACGCTTGTCATCGTCGTCCTTGTAGTCCATAAGCT
TGGG-3′

The two strands were then annealed into duplex according to manufacturer's protocol and inserted into pcDNA3.0 mammalian expression vector following HindIII and Not I double digestion (HindIII, Thermo Scientific, FD0504; Not I, Thermo Scientific, FD0594). The FLAG-βsyn plasmid was constructed by mutating the CGA residues (corresponding to the first arginine residue in the “RRRG” degron peptide sequence) into the stop codon TGA on the FLAG-βsyn-degron plasmid. FLAG-βsyn point mutation primers (synthesized by IDT): forward: 5′-TATTTGGTTGGTTGACGACGAGGCT-3′; reverse: 5′-AGCCTCGTCGTCAACCAACCAAATA-3′. HA-β-synuclein and HA-γ-synuclein were PCR amplified from rat cDNA library and then inserted into pcDNA3.0 mammalian expression vector following BamHI (Thermo Scientific, FD0054) and Not I double digestion.

HA-β-synuclein primers (synthesized by IDT):
forward:
5′-CGGGATCCATGTACCCATACGATGTTCCAGATTACGCTATGGACGT
GTTCATGAAGGGCCTGTCCATG-3′
reverse:
5′-AAGGAAAAAAGCGGCCGCTTACGCCTCTGGCTCGTATTCCTGATAT
TCCTC-3′
HA-γ-synuclein primers (synthesized by IDT):
forward:
5′-CGGGATCCATGTACCCATACGATGTTCCAGATTACGCTATGGACGT
CTTCAAGAAAGGCTTCTCCATT-3′
reverse:
5′-AAGGAAAAAAGCGGCCGCCTAGTCTCCTCCACTCTTGGCCTCTTCG
CCCTC-3′

All plasmid sequences were verified by DNA sequencing.

### HEK 293 Cell Culture and Plasmid Transfection

Human Embryonic Kidney 293 (HEK 293) cells that are commonly used for plasmid transfection and gene expression were purchased from ATCC (ATCC® CRL-1573™) and no mycoplasma contamination were observed during the experiment. HEK 293 cells were cultured in Dulbecco's Modified Eagle's Medium (DMEM) (Sigma, D6429) supplemented with 10% Fetal Bovine Serum (FBS) (Invitrogen, 12483020). When HEK 293 cells achieved 90% confluence, plasmids were transfected into the cells using Lipofectamine 2000 (Invitrogen, 11668019) according to manufacturer's instruction. Total plasmid transfection amount in every group was made equal by supplementing pcDNA3.0 empty vector. HEK 293 cells were then maintained in the 37° C. incubator with 95% O₂ and 5% CO₂ for 48 hrs before being used in experiments.

### Biacore Peptide-Protein Binding Assay

Biacore experiments were performed using a Biacore 3000 instrument (GE Healthcare Biosciences, Upsala, Sweden) and HBS running buffer, pH 7.4, containing 10 mM HEPES, 150 mM NaCl, 3 mM EDTA, and 0.005% surfactant P20. A research-grade CM5 sensor chip was activated with a mixture containing equal molar amounts of EDC (N-ethyl-N′-(dimethylaminopropyl) carbodiide and NHS (N-hydroxysuccinimide). Purified recombinant human α-synuclein (N-terminal histidine tagged, Sigma, S7820-500UG), diluted in sodium acetate pH 3.1, was then injected and covalently coupled to a flow cell of the sensor chip surface by amide bonding. Residual unreacted sites were blocked with ethanolamine. A reference surface, to account for non-specific binding, was similarly generated by activating and blocking an adjacent flow cell. Approximately 1500 resonance units (RU), equivalent to a surface concentration of 1500 pg/mm² of α-synuclein, was immobilized.

To ascertain the binding interaction between α-synuclein knockdown peptides and α-synuclein, synthetic Tat-βsyn-degron peptide or control tat peptide was serially diluted and sequentially injected over the active surface containing immobilized α-synuclein and the reference surface for 3 minutes at a flow rate of 30 μl/minute. The peptides were then allowed to dissociate for 3 minutes during which time HBS running buffer was injected. The resultant sensorgrams were double-referenced by subtracting out the binding on the reference surface and the response from the HBS blank buffer control. Binding response report points were collected 20 seconds into the dissociation phase of the interaction at time 200 seconds, which represents a stable binding response and excludes bulk refractive index changes and nonspecific binding.

### Primary Neuron Culture

Primary rat neuron cultures were prepared from embryos of pregnant Sprague-Dawley rats (E18). The experimental protocol was approved by the University of British Columbia Animal Care Committee. Briefly, the cortical tissue or ventral midbrain tissue was isolated into ice cold HBSS (Invitrogen, 14170-112) and then digested with 0.25% trypsin-EDTA at 37° C. for 30 min. After washing with warm DMEM (supplemented with 10% FBS) three times, neurons were suspended in neuron culture media and dissociated by trituration using varying sizes of pipettes. Neurons were then centrifuged, and the pellet was re-suspended in culture media, washed twice with culture media, and plated on the poly-D-lysine-coated plates. Neuron culture was maintained in the 37° C. incubator with 95% O₂ and 5% CO₂. The morning after culturing, 2/3 of the neuron culture media was replaced with fresh neuron culture media. Media was then replaced every 3-4 days. Primary rat cortical neuron culture was used 14 days in vitro (DIV) and primary rat ventral midbrain neuron culture was used 3 DIV.

### Peptide and MPP+ Treatment

Tat-βsyn-degron and Tat-βsyn peptides were first dissolved in sterile water as a 25 mM stock solution and then diluted directly in the neurobasal culture media to make the desired working concentration. 20 mM MPP+iodide stock solution was made freshly each time and diluted in the neurobasal culture media directly to make the desired working concentration. For 48 hrs treatment, neuron culture media containing MPP+ and peptides were replaced every 24 hrs.

### Cytotoxicity

DIV 14 primary cortical neurons were treated with different doses of the Tat-βsyn-degron and Tat-βsyn peptides and culture media were collected 24 hours after peptide treatment to measure cytotoxicity using a LDH assay kit (Roche, 11644793001). The culture medium from cells treated with 2% Triton X-100 for 30 min at 37° C. was used as the positive assay control and the culture medium from untreated cells were used as the negative assay control.

### TH-Positive Neuron Counting In Vitro

Neurons were rinsed 4× with ice-cold PBS, 2 min each time, and fixed with 4% PFA for 1 hr at 37° C. Neurons were then washed 3×5 min in PBS with gentle agitation, and subsequently incubated in 0.25% TritonX-100/PBS for 5 min at room temperature with gentle shaking. Next, neurons were washed 1× in PBS for 5 min, and then incubated for 30 min at 37° C. in 10% BSA/PBS without agitation to block non-specific staining. To label TH, neurons were incubated in primary TH antibody (1:100 dilution in 3% BSA/PBS) at 4° C. for 5 days without agitation.

Neurons were then washed 6×2 min in PBS, and incubated in Alexa Fluor 488 (Life technologies, A-11034; 1:500 dilution in 3% BSA/PBS) for 45 min at 37° C. without agitation. Next, neurons were washed 6×2 min in PBS, mounted on glass slides with Fluoromount-G slide mounting media (SouthernBiotech, 0100-01) and stored at room temperature overnight to dry. Neurons were then imaged with the Zeiss Axio Observer D1 microscope at 20× and 10 fields of view per coverslip were randomly selected and counted. Imaging and counting for TH-positive cells were performed by an experimenter blinded to the treatment conditions.

### Pre-Formed Fibrils (PFFs)

PFFs were generated in-house based on the Volpicelli-Daley et al protocol. Synuclein monomers were shaken at 37° C. at 1000 rpm for 7 days. PFFs were sonicated with 60 pulses at 10% power (total of 30 s, 0.5 s on, 0.5 s off; FB120, Fisher Scientific), stored at −80° C., and kept at room temperature during the intracerebral injections. PFFs were characterized using a negative staining protocol and analyzed using an electron microscope. PFFs were added to 200 mesh cupper carbon grid (3520C-FA, SPI Supplies) and stained with 2% acetate uranyl (22400-2, EMS). PFFs were visualized using a transmission electron microscope (FEI Tecnai 12 Bio Twin 120 kV TEM) coupled to a AMT XR80C CCD camera, and analyzed with ImageJ 1.5 and Matlab 2017b software.

### Intracerebral Injection in M83 Mice

M83 mice were anesthetized with 2% isoflurane and underwent stereotaxic injection with one of the following inoculants: human α-synuclein pre-formed fibrils (PFFs; total protein concentration, 12.5 μg per brain) or phosphate buffer saline (PBS), at a rate of 0.25 μl per min (for a total volume of 2.5 μl). A dose of 20 mg/kg carprofen and 250 mg/ml bupivacaine were administered subcutaneously to the mouse prior to the craniotomy. 5 μl Hamilton syringe with a 33-gauge needle was placed unilaterally in the right dorsal striatum (+0.2 mm relative to bregma, +2.0 mm lateral from midline, and 2.6 mm ventral from skull dorsal surface) using the Mouse Brain in Stereotaxic Coordinates Atlas. Mice received intraperitoneal injections of PBS, the Tat-βsyn-degron peptide (40 mg/kg) or the Tat-βsyn peptide (40 mg/kg), daily for 12 days, including 3 days before and 9 days after the intracerebral (i.e.) injection, and subsequently every other day for 8 days (20 days in total, FIG. 5a).

### The Enzyme-Linked Immunosorbent Assay (ELISA)

Brains were lysed (1 g tissue per 3 mL solution) in a buffer solution (50 mM Tris pH 8, 150 mM NaCl, 5 mM EDTA, 1% NP-40, 0.5% sodium deoxycholate, 0.1% SDS, protease and inhibitor cocktail [aprotinin, leupeptin and benzamidine], and phosphatase inhibitor cocktail) on ice. Lysates were homogenized at 1,600 g several times, sonicated and finally centrifuged at 100,000 g for 30 min. The supernatant was collected and processed with the Human α-synuclein ELISA kit (KHB0061, ThermoFisher Scientific) and analyzed with a Tecan microplate reader (Tecan Infinite M200 Pro, life science). The level of α-synuclein was expressed as ng/ml total sample.

### Mouse MPTP In Vivo Model

C57BL/6 mice (20-25 g) received intraperitoneal (i.p.) injection of 30 mg/kg parkinsonian toxin MPTP hydrochloride once a day for 5 days to induce dopaminergic neuron death in the substantia nigra, while the control mice received equal volumes of saline injection. 6 μmol/kg Tat-βsyn-degron peptide or control Tat-βsyn peptide was i.p. injected into the MPTP-treated mice every 12 hrs from the first day of MPTP injection until 7 days after the last injection of MPTP. All groups of mice then underwent a rotarod test before they were sacrificed.

### Behavioral Tests

The rotarod test was performed as previously described (Heldermon, C. D. et al., PloS one 2, e772, doi:10.1371/journal.pone.0000772 (2007)), with modifications. Briefly, 6 days after the last MPTP injection, all mice received 4 rounds of training on the rotarod (Stoelting Co.). In the first two rounds of training, the rotarod was maintained at constant speed of 20 rpm for 3 min. In the second two rounds of training, the rotarod reversed rotation direction every 3 turns at the constant speed of 20 rpm for 3 min. 24 hrs after the last round of training, all groups of mice received formal rotarod testing in which the rotarod reversed rotation direction every 3 turns at the constant speed of 20 rpm. Mice were tested 10 times at 20 min intervals, and the time that they remained on the rotarod during each test was recorded. Maximum test time (cut-off limit) was 300s. The motor performance of the mouse was expressed as the latency to fall off the rotarod.

### Immunoblotting

Brain tissues or cultured cells were lysed on ice in the lysis buffer and then the solution was centrifuged at 14,000 rpm for 10 min at 4° C. Next, the supernatant was collected and protein concentrations were determined using a BCA protein assay kit (Thermo Scientific, 23227). Equal amount of protein samples was mixed with 4× sample buffer, boiled at 100° C. for 5 min, and separated on 10% sodium dodecyl sulphate-polyacrylamide gel electrophoresis (SDS-PAGE). Proteins were then transferred to Immobilon-PTM polyvinylidene fluoride (PVDF) membranes (Bio-Rad, 162-0177). The membranes were blocked with 5% non-fat milk in Tris-buffered saline containing 0.1% Tween-20 (TBST) for 1 hr at room temperature, and then incubated overnight at 4° C. with primary antibody. After washing 3×5 min in TBST, membranes were incubated with horseradish peroxidase-conjugated secondary antibody for 1 hr at room temperature. After another three washes with TBST, protein was visualized in the Bio-Rad Imager using ECL western blotting substrate (Pierce, 32016). The band density of each protein was quantified by the Bio-Rad Quantity One software and the relative optical density was analyzed relative to the loading control β-actin on the same membrane.

### Immunohistochemical Staining

C57BL/6 mice were anesthetized with 1.5 g/kg urethane (Sigma, U2500) and then perfused with 0.9% saline and 4% paraformaldehyde. Brains were removed and fixed in 4% paraformaldehyde for 24 hrs before being transferred to 30% sucrose/PBS solution for cryoprotection. The brains were then sectioned into 30 μm slices using a Leica cryostat. After blocking and permeabilization using a PBS solution containing 1% BSA and 0.2% Triton X-100 for 30 min at room temperature, the brain slices were incubated with anti-TH antibody (1:800 dilution) for 3 days at 4° C. Finally, the TH-positive neurons in the substantia nigra and TH-positive neuronal terminals in the striatum were stained using the anti-mouse Ig HRP detection kit (BD Transduction Laboratories, 551011) according to manufacturer's instruction, and visualized under Zeiss Axio Observer D1 microscope.

Brains from M83 mice that received i.e. and i.p. injections were processed for immunohistochemistry. Mice were anesthetized with 2% isoflurane and were perfused with 10% formalin intracardially. The brains were removed and post-fixed with 10% formalin for 24 hrs at 4° C. Coronal sections were cut with a paraffin microtome at 5 μm thickness. Tissue sections were incubated in citrate buffer pH 6.0 for 10 minutes, then rinsed with TBST and incubated in 3% oxidase peroxidase for 15 minutes. Subsequently, the sections were blocked with 10% normal goat serum in TBST for 30 minutes at room temperature and incubated with the following primary antibodies: anti phosphorylated pS129 α-synuclein (1:500 dilution) or anti-Iba1 (1:250 dilution) in 5% normal goat serum in TBST, overnight at 4° C. Afterwards, the sections were incubated in secondary antibodies, goat anti-mouse-HRP (1:500 dilution) or goat anti-rabbit-HRP (1:500 dilution), for 30 minutes at room temperature. The peroxidase reaction product was visualized as a brown precipitate by incubation of the tissue with the DAB substrate kit (8059, cell signal technology). Immunohistochemistry sections were examined by bright field microscope (Olympus DP-21SAL coupled to a digital camera DP21/DP26). Coronal sections were analyzed using Fiji-ImageJ 1.5 software to detect the total area labeled with the peroxidase immunoreaction product. The results were analyzed using GraphPad Prism software.

### Densitometric Analysis of Striatal TH Staining

The optical density of the TH-positive neuronal terminal staining in the mouse dorsolateral striatum where dopaminergic inputs from substantia nigra pars compacta were received was quantified using NIH Image J software. The optical density from the overlying corpus callosum was used as a background and subtracted from every measurement in the striatum. The optical density in the experimental group was normalized to the value from the control group.

### Dopaminergic Neuron Counting in the Mouse Substantia Nigra Pars Compacta

Mouse substantia nigra was sliced into a series of 30 μm sections (from rostral to caudal), and one in every six sections was stained with anti-TH antibody. The substantia nigra pars compacta was outlined using TH-positive neurons between identifiable landmarks⁴¹ from bregma −2.92 mm to −3.64 mm. Identifiable TH-positive neurons in three stained sections within this distance from each animal were manually counted by an experimenter blinded to the treatment conditions under the Zeiss Axio Observer D1 microscope at 40×, and the total number of TH-positive neurons in this region was estimated using the following equation: N=counted number×6.

### Statistical Analysis and Reproducibility

Cell cultures and animals were randomly chosen for experiments and the data was analyzed by SPSS software and expressed as mean±S.E.M. Bar graphs were shown in dot-plot format in order to show data distribution. Measurements were taken from distinct samples for statistical analysis. Data were analyzed by one-way ANOVA, two-way ANOVA or unpaired t test, depending on the experiment condition. ANOVA analyses were followed by Bonferroni or Tukey's HSD post hoc test, depending on the number of groups under investigation. The sample sizes and number of replicates were indicated in the figure legends. *P≤0.05, **P≤0.01, and ***P≤0.001 were considered as significant differences.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent, or patent application was specifically and individually indicated to be incorporated by reference in its entirety.

Incorporated by reference into this specification is the text-based computer-readable format sequence listing in a file named “092409_00019_ST25_SL_ST25_June2025.txt”, with this file having been created on Jun. 10, 2025 with a file size of 3,111 bytes.

1. A method of treating a disease characterized by abnormal aggregation of α-synuclein in a subject, the method comprising administering to the subject a therapeutically effective amount of a peptide comprising an α-synuclein binding domain operably linked to a protein transduction domain and a proteasomal targeting domain, wherein the α-synuclein binding domain is derived from a reversed sequence of β-synuclein.

2. (canceled)

3. (canceled)

4. The method of claim 1, wherein abnormal aggregation of α-synuclein in one or more regions of the brain of the subject is reduced.

5. The method of claim 1, wherein inflammation in one or more regions of the brain of the subject is reduced.

6. The method of claim 1, wherein one or more symptoms of the disease are ameliorated.

7. The method of claim 1, wherein the binding affinity of the peptide for α-synuclein is significantly greater than the binding affinity of the peptide for: (i) β-synuclein; or (ii) γ-synuclein.

8. The method of claim 1, wherein the α-synuclein binding domain comprises an amino acid sequence substantially identical to SEQ ID NO: 2.

9. The method of claim 1, wherein the protein transduction domain is selected from the group consisting of:
a. the HIV Tat transduction domain, YGRKKRRQRRR;
b. the Drosophila melanogaster Antennapedia domain Antp (amino acids 43-58), RQIKWFQNRRMKWKK;
c. Buforin II, TRSSRAGLQFPVGRVHRLLRK;
d. hClock-(amino acids 35-47) (human Clock protein DNA-binding peptide), KRVSRNKSEKKRR;
e. MAP (model amphipathic peptide), KLALKLALKALKAALKLA;
f. K-FGF, AAVALLPAVLLALLAP;
g. Ku70 derived peptide, comprising a peptide selected from the group comprising VPMLKE, VPMLK, PMLKE or PMLK;
h. Prion, Mouse Prpe (amino acids 1-28), MANLGYWLLALFVTMWTDVGLCKKRPKP;
i. pVEC, LLIILRRRIRKQAHAHSK;
j. Pep-I, KETWWETWWTEWSQPKKKRKV;
k. SynB1, RGGRLSYSRRRFSTSTGR;
l. Transportan, GWTLNSAGYLLGKINLKALAALAKKIL;
m. Transportan-10, AGYLLGKINLKALAALAKKIL;
n. CADY, Ac-GLWRALWRLLRSLWRLLWRA-cysteamide;
o. Pep-7, SDLWEMMMVSLACQY;
p. FIN-1, TSPLNIHNGQKL;
q. VT5, DPKGDPKGVTVTVTVTVTGKGDPKPD; or
r. pISL, RVIRVWFQNKRCKDKK.

10. The method of claim 1, wherein the protein transduction domain is the HIV Tat transduction domain YGRKKRRQRRR.

11. The method of claim 1, wherein the proteasomal targeting domain comprises a degron.

12. The method of claim 11, wherein the degron comprises the amino acid sequence RRRG.

13. The method of claim 1, wherein the disease is selected from Parkinson's disease, diffuse Lewy body disease, transitional Lewy body dementia, and multiple system atrophy.

14. The method of claim 13, wherein the disease is Parkinson's disease.

15. The method of claim 1, wherein the subject is a human.

16. The method of claim 1, wherein the administration of the peptide is by systemic administration.

17. The method of claim 16, wherein the systemic administration is intravenous administration.

18. The method of claim 1, wherein the peptide comprises an amino acid sequence having at least about 90% sequence identity to SEQ ID NO: 1.

19. The method of claim 1, wherein the peptide comprises an amino acid sequence having at least about 95% sequence identity to SEQ ID NO: 1.

20. The method of claim 1, wherein the peptide comprises the amino acid sequence of SEQ ID NO: 1.

21.-41. (canceled)

42. A pharmaceutical composition for administration to a subject having a disease that is, or is characterized by, a synucleinopathy, the pharmaceutical composition comprising:
a. a therapeutically effective amount of a peptide comprising an α-synuclein binding domain operably linked to a protein transduction domain and a proteasomal targeting domain, wherein the α-synuclein binding domain is derived from a reversed sequence of β-synuclein; and
b. a carrier.

43. The pharmaceutical composition of claim 42, wherein the peptide comprises an amino acid sequence having at least about 90% sequence identity to SEQ ID NO: 1.

44. The pharmaceutical composition of claim 42, wherein the peptide comprises an amino acid sequence having at least about 95% sequence identity to SEQ ID NO: 1.

45. The pharmaceutical composition of claim 42, wherein the peptide comprises the amino acid sequence of SEQ ID NO: 1.

46. The pharmaceutical composition of claim 42, wherein the composition is for systemic administration.

47. The pharmaceutical composition of claim 46, wherein the composition is for intravenous administration.

48. A pharmaceutical composition for administration to a subject having a disease that is, or is characterized by, a synucleinopathy, the pharmaceutical composition comprising:
a. a therapeutically effective amount of a polynucleotide encoding for a peptide, wherein the peptide comprises an α-synuclein binding domain operably linked to a protein transduction domain and a proteasomal targeting domain, and wherein the α-synuclein binding domain is derived from a reversed sequence of β-synuclein; and
b. a carrier.

49. The pharmaceutical composition of claim 48, wherein the peptide comprises an amino acid sequence having at least about 90% sequence identity to SEQ ID NO: 1.

50. The pharmaceutical composition of claim 48, wherein the peptide comprises an amino acid sequence having at least about 95% sequence identity to SEQ ID NO: 1.

51. The pharmaceutical composition of claim 48, wherein the peptide comprises the amino acid sequence of SEQ ID NO: 1.

52. The pharmaceutical composition of claim 48, wherein the polynucleotide comprises the amino acid sequence of SEQ ID NO: 9.


SEQUENCE LISTING (9 sequences)

SEQ ID NO: 1 - protein, 25 aa, Artificial Sequence
  The sequence is synthesized (YGRKKRRQRR RRTKSGVYLV GRRRG)
YGRKKRRQRRRRTKSGVYLVGRRRG

SEQ ID NO: 2 - protein, 10 aa, Artificial Sequence
  The sequence is synthesized (RTKSGVYLVG)
RTKSGVYLVG

SEQ ID NO: 3 - protein, 10 aa, Artificial Sequence
  The sequence is synthesized (GVLYVGSKTR)
GVLYVGSKTR

SEQ ID NO: 4 - DNA, 79 bp, Artificial Sequence
  The sequence is synthesized (AGCTTATGGACTACAAG GACGACGATGACAAGGGGGTGCTGTACGTGGGGAGCAAGACGAGGCGACGACGAGGCTAAGC)
agcttatggactacaaggacgacgatgacaagggggtgctgtacgtggggagcaagacgaggcgacgacgaggctaagc

SEQ ID NO: 5 - DNA, 79 bp, Artificial Sequence
  The sequence is synthesized (GGCCGCTTAGCCTCGTC GTCGCCTCGTCTTGCTCCCCACGTACAGCACCCCCTTGTCATCGTCGTCCTTGTAGTCCATA)
ggccgcttagcctcgtcgtcgcctcgtcttgctccccacgtacagcacccccttgtcatcgtcgtccttgtagtccata

SEQ ID NO: 6 - DNA, 99 bp, Artificial Sequence
  The sequence is synthesized (CCCAAGCTTATGGACTA CAAGGACGACGATGACAAGCGTACTAAATCTGGTGTTTATTTGGTTGGTCGACGACGAGGCTAAG CGGCCGCTTTTTTCCTT)
cccaagcttatggactacaaggacgacgatgacaagcgtactaaatctggtgtttatttggttggtcgacgacgaggctaagcggccgcttttttcctt

SEQ ID NO: 7 - DNA, 99 bp, Artificial Sequence
  The sequence is synthesized (AAGGAAAAAAGCGGCCG CTTAGCCTCGTCGTCGACCAACCAAATAAACACCAGATTTAGTACGCTTGTCATCGTCGTCCTTG TAGTCCATAAGCTTGGG)
aaggaaaaaagcggccgcttagcctcgtcgtcgaccaaccaaataaacaccagatttagtacgcttgtcatcgtcgtccttgtagtccataagcttggg

SEQ ID NO: 8 - DNA, 42 bp, Artificial Sequence
  The sequence is synthesized (GGGGTGCTGTACGTGGG GAGCAAGACGAGGCGACGACGAGGC)
ggggtgctgtacgtggggagcaagacgaggcgacgacgaggc

SEQ ID NO: 9 - DNA, 42 bp, Artificial Sequence
  The sequence is synthesized (CGTACTAAATCTGGTGT TTATTTGGTTGGTCGACGACGAGGC)
cgtactaaatctggtgtttatttggttggtcgacgacgaggc